(19) United States
(12) Patent Application Publication
(10) Pub. No.: US 2021/0170026 A1
(43) Pub. Date: Jun. 10, 2021

(54) ANTICANCER AGENT, RADIOSENSITIZER, AND FOOD COMPOSITION

(71) Applicants: KRACIE PHARMA, LTD., Tokyo (JP); NATIONAL CANCER CENTER, Tokyo (JP); TOKYO UNIVERSITY OF SCIENCE FOUNDATION, Tokyo (JP)

(72) Inventors: Hiroyasu ESUMI, Tokyo (JP); Katsuya TSUCHIHARA, Chiba (JP); Takanori KAWASHIMA, Toyama (JP)

(73) Assignees: KRACIE PHARMA, LTD., Tokyo (JP); NATIONAL CANCER CENTER, Tokyo (JP); TOKYO UNIVERSITY OF SCIENCE FOUNDATION, Tokyo (JP)

(21) Appl. No.: 16/317,358

(22) PCT Filed: Jul. 10, 2017
(86) PCT No.: PCT/JP2017/025071

§ 371 Date: Jan. 11, 2019

(30) Foreign Application Priority Data
Jul. 14, 2016 (JP) 2016-139112

(51) Int. Cl. A61K 41/00; A61K 31/7048; A61K 31/365; A23L 33/105; A23L 29/00; A61K 36/28; A61K 36/634

(52) U.S. Cl. CPC A61K 41/0038; A61K 31/7048; A61K 31/365; A61K 36/634; A23L 29/035; A61K 36/28; A23L 33/105

(57) ABSTRACT

Provided are an anticancer agent, radiosensitizer, and food composition capable of enhancing the effects of radiation therapy. The present inventors discovered that tumors shrink significantly in comparison to an untreated group and the respective monotherapy groups when treatment by burdock fruit extract containing arctigenin and radiation therapy were used in combination on mice transplanted with human pancreatic cancer cells. The anticancer agent of the present invention is an anticancer agent containing arctigenin and/or arctiin as the active ingredient, to be used in combination with radiation therapy. The arctigenin and/or arctiin may be contained as burdock, burdock fruit, burdock sprout, or forsythia, or an extract extracted from these.

### TECHNICAL FIELD

The present invention relates to an anticancer agent to be used in combination with radiation therapy, a radiosensitizer and a food composition for enhancing an effect of radiation therapy.

### BACKGROUND ART

The three major methods of cancer treatment are “surgery”, “treatment with an anticancer agent” and “radiation therapy”. Of these three, “radiation therapy” is a relatively new therapeutic method. Radiation therapy is a therapeutic method in which radiation such as x-rays, y-rays and electron beams is applied to cancer cells to kill the cancer cells. Radiation stops the division of the cancer cells or causes apoptosis of the cells by damaging the DNA of the cancer cells. Moreover, radiation activates oxygen in the cells into active oxygen, and the power of damaging DNA is thus enhanced. Radiation therapy has, for example, the following advantages: tissues are not removed by surgery; local treatment is possible; and the damage to healthy tissues is minor. Combination therapy using an anticancer agent and radiation is often used for cancers with a risk of aftereffects of surgery (such as laryngeal cancer, cervical cancer, bladder cancer and prostate cancer) or for pancreatic cancer which is difficult to treat with surgery. The purposes of radiation therapy are complete cure, life-prolonging treatment, palliative care and the like, and the purpose varies with the kind of cancer, the stage and the like.

Fifty to seventy percent of cancer patients receive radiation therapy in the process of treatment. Because radiation poses a risk of damaging not only tumor tissues but also healthy tissues, efforts are given to minimize the damage by limiting the radiation to the lesion or shielding the parts which should not be irradiated during treatment. Physical functions are deteriorated when radiation damages the cells of healthy tissues and causes cell death and when the influence is accumulated and spread, and the deterioration is called radiation injury (NPL 1). Each organ has a different degree of radiosensitivity, and the factor determining the degree is the nature of the constituting cells. Cells with a high frequency of cell division and a high number of future cell division rounds, namely cells with undifferentiated forms and functions, are highly sensitive. Thus, hematopoietic stem cells and somatic stem cells are easily affected, and the transient cytopenia and hair loss observed after exposure to a low dose are results thereof. In case of exposure to a high dose of radiation, characteristic disorders appear in the acute phase, depending on the radiosensitivity of the irradiated organ. Specifically, when blood-sending tissues and digestive tract, which have especially high radiosensitivity, are exposed to radiation of approximately 1.5 Gy and 5 Gy or more, respectively, disorders are caused, and exposure to radiation of several-dozen grays or more influences the central nerve system.

A damage caused when the energy of radiation is directly absorbed by the target molecule is called the direct effect of radiation (NPL 2). By the direct effect of radiation, the target molecule is excited or ionizes and becomes unstable due to extra energy. In the process of releasing the extra energy, the covalent bond of the target molecule is cut, and the target molecule turns into two radicals.

When a molecule other than the target absorbs the energy of radiation and forms an activator such as a radical, the activator reacts with the target molecule and causes a damage. Such a damage is called the indirect effect (NPL 2). In an aqueous solution, the energy of radiation is first absorbed by a water molecule, and a radical such as a hydroxyl radical, a hydrogen radical, a hydrated electron or hydrogen peroxide or a molecular product is generated. Such an activator works by moving in the water and causing chemical reaction with the target molecule.

The direct effect and the indirect effect can be distinguished from each other by the presence or absence of a dilution effect, a chemical protection effect, an oxygen effect and the like (NPL 2). In general, as the concentration of a substance in water becomes lower, the proportion of molecules inactivated by radiation increases. This phenomenon is called a dilution effect and indicates the indirect effect. Through application of a certain dose of radiation, the amount of an inactivated substance should decrease as the concentration of the target substance in water becomes lower if there is only the direct effect. When water molecules are decomposed by radiation and when some amounts of a radical and a molecular product are generated, however, certain amounts of the radical and the molecular product are generated at a certain dose. Thus, a certain amount of the substance is inactivated regardless of the concentration of the target substance in water. Thus, the dilution effect is observed.

The presence of oxygen changes the reaction of radiation therapy both qualitatively and quantitatively (NPL 2). One of the changes is to react with a hydrated electron or a hydrogen atom and generate a superoxide (e aq⁻+O₂→O₂⁻; H.+O₂→HO₂.). HO₂. has a potent oxidation power and generates hydrogen peroxide or an alkoxyl radical (RO.) with an instable organic molecule through reaction with an organic biomolecule such as nucleic acids, proteins and lipids.

For the above reasons, when x-rays or y-rays are applied under conditions with a high oxygen partial pressure, higher effects than those of irradiation under a low oxygen pressure can be obtained. This is called an oxygen effect, and its degree is indicated by an oxygen enhancement ratio (OER) which is defined by [the ratio of the dose necessary to cause a specific effect without oxygen to the necessary dose with oxygen]. This is believed to be because due to the presence of oxygen, a target molecule which has been directly or indirectly excited by radiation reacts with oxygen and generates a peroxide and because damage by radiation is thus fixed. That is, because the oxygen effect is less likely to be caused under a hypoxic condition, resistance to radiation is caused in comparison in tissues under a normal oxygen partial pressure (about 40 mmHg), and the dose necessary for obtaining a certain radiation effect becomes higher than that for healthy tissues. This is a cause for weakening the effects of radiation therapy.

It has been reported that the cancer microenvironment of especially pancreatic cancer, of various types of cancers, is a hypoxic and poor nutrition environment and that the conventional anticancer agents and radiation are less effective. When pancreatic cancer is treated with radiation, it is necessary to use a radiosensitizer or to change the cancer microenvironment into an environment in which the oxygen effect is easily caused. An example of known radiosensitizers is broxuridine, which is incorporated into the DNA instead of thymidine during the cell proliferation, and which enhances the sensitivity to radiation. Moreover, in order to enhances the sensitivity to radiation, there is a method called KORTUC, which enhances the oxygen effect by injecting oxydol, which decomposes antioxidative enzymes, and hyaluronic acid, which causes oxydol to stay in the affected part, into cancer. Broxuridine and the KORTUC method are sometimes used clinically but are not yet widely used in general cases.

PTL 1 discloses a radiosensitizer which protects healthy cells during radiation therapy and at the same time increases the radiosensitivity of tumor cells. The radiosensitizer contains ascorbic acid, a pharmaceutically acceptable salt of ascorbic acid or a pharmaceutically acceptable solvate of ascorbic acid as the active ingredient.

### CITATION LIST

### Patent Literature

PTL 1: JP-A-2014-139138

### Non Patent Literature

NPL 1: “Therapeutic Strategy using Natural Immunity to Organ Disorders Caused by Radiation-Induced Cell Death”, Experimental Medicine, Japan, 2016, Vol. 34, No. 7, pp 110-115
NPL 2: “Direct Effect and Indirect Effect of Radiation” [online], Research Organization for Information Science and Technology, (searched on Jun. 27, 2016), internet <URL: http://www.rist.or.jp/atomica/data/dat_detail.php?Title_No=09-02-02-10>

### SUMMARY OF INVENTION

### Technical Problem

The cancer microenvironment of pancreatic cancer or the like is in short of blood vessels and is an extremely hypoxic and extremely poor nutrition environment. In such an environment, the conventional anticancer agents and radiation are less effective, and cancer stem cells involving in the malignant transformation of cancer are easily generated. Thus, development of a method capable of enhancing the effects of radiation therapy is desired.

An object of the invention is to provide an anticancer agent, a radiosensitizer and a food composition capable of enhancing an effect of radiation therapy.

### Solution to Problem

The present inventors have discovered that tumors shrink significantly in comparison to an untreated group and the respective monotherapy groups when treatment by burdock fruit extract containing arctigenin and radiation therapy were used in combination on mice transplanted with human pancreatic cancer cells. The inventors have discovered that arctigenin inhibits the oxygen consumption in a tumor tissue, increases the blood flow in the tumor and thus improves the hypoxic environment of the tumor, thereby enhancing the effects of radiation therapy.

The invention provides an anticancer agent to be used in combination with radiation therapy containing arctigenin and/or arctiin as an active ingredient.

The invention also provides the above anticancer agent, wherein the arctigenin and/or arctiin is contained as burdock, burdock fruit, burdock sprout, forsythia or an extract extracted from these.

The invention also provides a radiosensitizer containing arctigenin and/or arctiin as an active ingredient.

The invention also provides a food composition for enhancing an effect of radiation therapy containing arctigenin and/or arctiin as an active ingredient.

The invention also provides the above food composition, wherein the arctigenin and/or arctiin is contained as burdock, burdock fruit, burdock sprout, forsythia or an extract extracted from these.

The invention also provides a pretreatment agent for radiation therapy containing arctigenin and/or arctiin as an active ingredient.

### Advantageous Effects of Invention

According to the invention, the effects of radiation therapy can be enhanced by improving the hypoxic environment of a tumor. Thus, when used in combination with radiation therapy, the invention can enhance the effects of radiation therapy, enhance the antitumor effect and further increase the survival rate.

### BRIEF DESCRIPTION OF DRAWINGS

FIG. 1 A figure explaining the test method used in Test Example 1.

FIG. 2 A graph showing the average tumor sizes of the untreated group and the arctigenin treatment group after two weeks of the first arctigenin treatment (before the radiation therapy).

FIG. 3 A graph showing the results of evaluation of the intratumoral hypoxic regions (photon counts/tumor volume) after the arctigenin treatment using IVIS.

FIG. 4 A figure showing the results of IVIS imaging of the untreated group and the arctigenin treatment group.

FIG. 5 A graph showing the changes in average tumor size (mean±S.E.) of the treatment groups.

FIG. 6 A box plot showing the tumor sizes of the treatment groups after the end of the treatment (two weeks after the radiation therapy).

FIG. 7 A box plot showing the tumor weights (g) of the treatment groups after the end of the treatment (two weeks after the radiation therapy).

FIG. 8 A figure showing the tumor states of the treatment groups after the end of the treatment (two weeks after the radiation therapy).

FIG. 9 A figure showing the tumor states of the treatment groups after the end of the treatment (two weeks after the radiation therapy).

FIG. 10 A figure explaining the test method used in Test Example 2.

FIG. 11 A graph showing the changes in average tumor size of the treatment groups.

FIG. 12 A figure showing the correlation between the intratumoral hypoxic region after the end of the treatment (two weeks after the radiation therapy) evaluated using IVIS and the tumor size (%) two weeks after the radiation therapy relative to the tumor size before the radiation therapy.

### DESCRIPTION OF EMBODIMENTS

The invention provides an anticancer agent to be used in combination with radiation therapy containing arctigenin and/or arctiin as an active ingredient.

The “radiation therapy” in this description is a therapeutic method for applying radiation to cancer cells and thus killing the cancer cells. The radiation used for the radiation therapy is not particularly limited as long as it is radiation which contributes to inhibition of proliferation or metastasis of cancer cells, killing of cancer cells or inhibition of development of cancer cells. The radiation may act on cancer cells either directly or indirectly. The radiation used for the radiation therapy is not limited but may be, for example, x-rays, γ-rays, electron beams or the like. The method for applying the radiation is not limited but may be, for example, stereotactic radiotherapy, high precision radiotherapy or the like.

When used in combination with radiation therapy, the anticancer agent of the invention can enhance an effect of the radiation therapy, enhance the antitumor effect, reduce a side effect of the radiation therapy and further increase the survival rate.

In this description, that treatment with the anticancer agent and radiation therapy are “used in combination” means that the anticancer agent is administered before the radiation therapy, together with the radiation therapy and/or after the radiation therapy. The order of administration and the dosage forms are not limited as long as the treatment with the anticancer agent and the radiation therapy are both used.

The side effects of radiation therapy are effects which do not meet the purposes of the treatment or which are unfavorable for the living body and include generally recognized side effects. Examples of the side effects of radiation therapy include symptoms such as skin inflammation including erosion or the like, mucosal inflammation, hair loss, diarrhea, anorexia, general malaise, pain, dyspnea, nausea, vomiting, fever, anosmia, organ dysfunction, interstitial pneumonia, organ failure and myelosuppression. Emotional distress accompanying such a physical disorder, such as anxiety, impatience, loss of interest, torpor, insomnia, sense of alienation, fear, adjustment disorder, depression and delirium, is also included in the side effects of radiation therapy. The anticancer agent of the invention can reduce especially skin inflammation such as erosion.

Arctigenin and arctiin are diphenylpropanoids (lignans) contained in plants such as burdock. Arctiin is a precursor of arctigenin and is known to be metabolized in the living body and converted into arctigenin. The anticancer agent of the invention may contain arctigenin or arctiin only or contain both arctigenin and arctiin.

As arctigenin and/or arctiin, chemically synthesized arctigenin and/or arctiin may be used, and arctigenin and/or arctiin isolated from a plant may also be used. Moreover, as arctigenin and/or arctiin, a plant containing arctigenin and/or arctiin itself or an extract of a plant may be used. Examples of plants containing arctigenin and/or arctiin include Arctium lappa (burdock) (sprouts, leaves, rhizomes and burdock fruit), Forsythia×intermedia (flowers, leaves, fruits and rhizomes), Forsythia viridissima var. koreana (flowers, leaves, fruits and rhizomes), Forsythia suspensa (forsythia) (flowers, leaves, fruits and rhizomes), Forsythia viridissima (flowers, leaves, fruits and rhizomes), Carthamus tinctorius, Centaurea cyanus, Cirsium vulgare, Centaurea benedicta (Cnicus benedictus), Cynara cardunculus, Onopordum acanthium, thistle (Aniurokoazami), Sesamum indicum, Ipomoea cairica, Polygala chinensis, Trachelospermum asiaticum var. glabrum, Trachelospermum asiaticum, Trachelospermum gracilipes var. liukiuense, Trachelospermum gracilipes, Trachelospermum jasminoides, Trachelospermum jasminoides var. pubescens, Wikstroemia indica, Persicaria pilosa, Cerasus jamasakura, Arabidopsis thaliana, amaranth, Juglans, Avena sativa, Triticum spelta, soft wheat, Cupressus lusitanica and Torreya nucifera. Of these examples, burdock (in particular, burdock fruit and burdock sprout) and forsythia (in particular, leaves) are preferable because the arctigenin and/or arctiin contents are high.

When an extract of a plant is used as arctigenin and/or arctiin, the extract may be prepared from the plant, for example, by the following method. For example, the extract used in the invention may be taken from a plant containing arctigenin and/or arctiin by two stages, namely an enzymatic conversion step and an extraction step using an organic solvent.

The enzymatic conversion step is a step of enzymatically converting arctiin contained in a plant into arctigenin by β-glucosidase, which is an endogenous enzyme of the plant. Specifically, by drying and cutting the plant and then keeping the plant at an appropriate temperature, endogenous β-glucosidase is caused to act, and the reaction from arctiin to arctigenin is advanced. For example, by adding a solution such as water to the cut plant and stirring the plant at a temperature of around 30° C. (20 to 50° C.) or by another procedure, the plant can be kept at a certain temperature.

The extraction step using an organic solvent is a step of extracting arctigenin and arctiin from a plant using an appropriate organic solvent. That is, this is a step of extracting an extract from the plant by adding an appropriate solvent after the arctigenin content of the plant has become high through the enzymatic conversion step. For example, an extract is taken by adding an appropriate solvent to the plant and heating and stirring for an appropriate period of time. The extract can be taken using any extraction method known to one skilled in the art, such as refluxing by heating, drip extraction, immersion extraction or a pressurized extraction method, in addition to heating and stirring.

Because arctigenin is insoluble in water, the yield of arctigenin can be improved by adding an organic solvent. As the organic solvent, any organic solvent can be used. For example, alcohols such as methanol, ethanol and propanol and acetone can be used. In view of the safety, 30% ethanol is preferably used as the organic solvent in the production method of the extract used for the anticancer agent of the invention. When the solvent is evaporated from the extract, a paste-like concentrate is obtained, and a dried product can be obtained by further drying the concentrate.

The anticancer agent of the invention may contain arctigenin and arctiin at an arctigenin/arctiin ratio by weight of 0.7 or more. The arctigenin/arctiin ratio by weight is not particularly limited but may be 1.3 or less. The anticancer agent of the invention may contain a plant extract containing arctigenin and arctiin at a ratio by weight of arctigenin/arctiin=0.7 to 1.3, for example a burdock fruit extract. Moreover, the anticancer agent of the invention may contain a burdock fruit extract containing 3% arctigenin or more. Such a burdock fruit extract can be obtained by the method for producing a burdock fruit extract described below. The anticancer agent of the invention contains a burdock fruit extract obtained by the method for producing a burdock fruit extract described below and thus can exhibit a stronger anticancer effect than that of an agent containing a conventional burdock fruit extract.

The anticancer agent of the invention can be a pharmaceutical preparation in any form. For example, the anticancer agent of the invention as an orally-administered pharmaceutical preparation can be: a tablet such as sugar-coated tablets, buccal tablets, coating tablets and chewable tablets; a troche; a pill; powder; a capsule including hard capsules and soft capsules; granules; a liquid such as suspensions, emulsions, syrups and elixirs; or the like.

In addition, the anticancer agent of the invention can be a pharmaceutical preparation for parenteral administration such as intravenous injection, subcutaneous injection, intraperitoneal injection, intramuscular injection, transdermal administration, nasal administration, transpulmonary administration, enteral administration, buccal administration and transmucosal administration. For example, the anticancer agent of the invention can be an injection, a transdermal absorbing tape, an aerosol, a suppository or the like.

The anticancer agent of the invention can be in a form suitable for foods and may be, for example, a solid, a liquid, granules, grains, powder, capsules, a cream, a paste or the like.

The anticancer agent of the invention can further contain any component which is generally used for pharmaceutical products, quasi drugs and foods. For example, the anticancer agent of the invention may further contain a base, a carrier, an excipient, a binder, a disintegrating agent, a lubricant and a coloring agent which are pharmaceutically acceptable and the like.

Examples of the carrier and the excipient used for the anticancer agent of the invention include lactose, glucose, white soft sugar, mannitol, dextrin, potato starch, corn starch, calcium carbonate, calcium phosphate, calcium sulfate, crystalline cellulose and the like.

Examples of the binder include starch, gelatin, syrup, tragacanth gum, polyvinyl alcohol, polyvinyl ether, polyvinylpyrrolidone, hydroxypropylcellulose, methylcellulose, ethylcellulose, carboxymethylcellulose and the like.

Examples of the disintegrating agent include starch, agar, gelatin powder, crystalline cellulose, calcium carbonate, sodium hydrogen carbonate, sodium alginate, sodium carboxymethylcellulose, calcium carboxymethylcellulose and the like.

Examples of the lubricant include magnesium stearate, hydrogenated vegetable oil, talc, macrogol and the like. As the coloring agent, any coloring agent which has been approved to be added to pharmaceutical products, quasi drugs and foods can be used.

In addition, the anticancer agent of the invention, if required, may be coated with one or more layers of white soft sugar, gelatin, refined shellac, gelatin, glycerin, sorbitol, ethylcellulose, hydroxypropylcellulose, hydroxypropylmethylcellulose, polyvinylpyrrolidone, phthalic acid cellulose acetate, hydroxypropylmethylcellulose phthalate, methyl methacrylate, a methacrylic acid polymer or the like.

Moreover, if required, a pH-controller, a buffering agent, a stabilizer, a preservative, an antiseptic, a diluent, a coating agent, a sweetener, an aroma, a solubilizing agent and the like may be added to the anticancer agent of the invention.

The arctigenin and/or arctiin content of the anticancer agent of the invention is an amount which can exhibit the effect of enhancing an effect of radiation therapy and can be appropriately set depending on the subject of application, the purpose and the administration method (intake method). For example, when the anticancer agent is orally taken by a human, arctigenin and/or arctiin can be preferably contained in a manner that the daily intake becomes 10 to 2000 mg.

The anticancer agent of the invention may be administered one to seven days a week although it is not particularly limited. For example, the anticancer agent of the invention may be administered every day or five or six times a week. Moreover, the anticancer agent of the invention may be administered before radiation therapy, together with radiation therapy and/or after radiation therapy.

For example, the anticancer agent of the invention may be administered at least once before radiation therapy and may be administered preferably for one week or longer, more preferably for two weeks or longer. When administered before radiation therapy, the anticancer agent of the invention can improve the hypoxic state of a tumor, improve the radiosensitivity of the tumor and heighten an effect of the radiation therapy. Moreover, by administering the anticancer agent before radiation therapy, a side effect of the radiation therapy can be inhibited.

Furthermore, the anticancer agent of the invention may be administered at least once after radiation therapy and may be administered preferably for one week or longer, more preferably for two weeks or longer. When administered after radiation therapy, the anticancer agent of the invention can heighten an effect of the radiation therapy. By administering the anticancer agent after radiation therapy, radiation injury due to the irradiation of radiation can be treated or improved.

The invention also provides a radiosensitizer containing arctigenin and/or arctiin as an active ingredient. In this description, the “radiosensitizer” is a drug which enhances an effect of radiation therapy. The radiosensitizer of the invention can be, for example, a radiosensitizer for improving the radiosensitivity of a tumor. The radiosensitizer of the invention improves the hypoxic state in a tumor and thus can improve the radiosensitivity of the tumor. The radiosensitizer of the invention can be composed in a similar manner to that of the anticancer agent described above.

The invention also provides a side effect-reducing agent for reducing a side effect of radiation therapy containing arctigenin and/or arctiin as an active ingredient. The drug of the invention can reduce, for example, a symptom caused by radiation therapy, such as skin inflammation including erosion or the like, mucosal inflammation, hair loss, diarrhea, anorexia, general malaise, pain, dyspnea, nausea, vomiting, fever, anosmia, organ dysfunction, interstitial pneumonia, organ failure and myelosuppression, and emotional distress caused by such a physical disorder, such as anxiety, impatience, loss of interest, torpor, insomnia, sense of alienation, fear, adjustment disorder, depression and delirium. “To reduce a side effect” in this description means that a side effect is reduced as a result and means, for example, not only that the drug acts directly on the cause of any of the symptoms described above and reduces the symptom but also that the drug improves the radiosensitivity of a tumor and thus indirectly reduces such a symptom. The drug of the invention can be composed in a similar manner to that of the anticancer agent described above.

The invention also provides a pretreatment agent for radiation therapy containing arctigenin and/or arctiin as an active ingredient. The pretreatment agent of the invention is a drug to be administered to a patient before radiation therapy. In this description, the “pretreatment agent for radiation therapy” means a drug which is administered to a patient before radiation therapy in order to heighten an effect of the radiation therapy or to reduce a side effect of the radiation therapy. The pretreatment agent of the invention can be composed in a similar manner to that of the anticancer agent described above.

The cancer microenvironment of pancreatic cancer or the like is in short of blood vessels and is an extremely hypoxic and extremely poor nutrition environment. Because the amount of oxygen which converts into active oxygen by radiation therapy is low in such an environment, radiation therapy is less effective. When administered to a cancer patient before radiation therapy, the pretreatment agent of the invention can inhibit the oxygen consumption of a tumor, increase the blood flow in the tumor and improve the hypoxic state. Thus, the pretreatment agent of the invention can improve the radiosensitivity in the tumor and heighten an effect of the radiation therapy. The pretreatment agent of the invention can also inhibit a side effect of the radiation therapy.

The treatment agent of the invention is administered at least once before radiation therapy. The treatment agent of the invention may be administered before radiation therapy continuously for a certain period and may be administered preferably for one week or longer, more preferably for two weeks or longer.

The invention also provides a food composition for enhancing an effect of radiation therapy containing arctigenin and/or arctiin as an active ingredient. The food composition of the invention can be composed in a similar manner to that of the anticancer agent described above.

The “food composition” in this description includes not only general foods and drinks but also foods for the ill, health foods, functional foods, foods for specified health use, dietary supplements, supplements and the like. Examples of the general foods and drinks include drinks, foods, processed foods, liquid foods (soups and the like), seasonings, nutrition-supplement drinks, snacks and the like. The “processed foods” in this description mean foods obtained by processing and/or cooking natural foodstuffs (meat, vegetables and the like) and include, for example, processed meat, processed vegetables, processed fruits, frozen foods, retort-pouched foods, canned foods, bottled foods, instant foods and the like. The food composition of the invention may be a food with an indication that an effect of radiation therapy is enhanced. The food composition of the invention may be provided in a form contained in a bag, a container or the like. The bag and the container used in the invention can be any bag and any container which are generally used for foods.

The invention also provides a method for enhancing an effect of radiation therapy including a step of administering arctigenin and/or arctiin to a patient. In this description, examples of the “effect of radiation therapy” include reduction in the tumor size and/or the tumor weight and the like. Arctigenin and/or arctiin may be administered to a patient in forms of the anticancer agent, the radiosensitizer and the like described above. The patient to which the method of the invention is applied is a patient in need of radiation therapy, for example, a cancer patient or the like. When the method of the invention is used, the radiosensitivity of a tumor can be improved.

The administration step in the method of the invention can be conducted before radiation therapy. The administration step may be conducted before radiation therapy at least once and may be conducted continuously for a certain period. The administration step may be conducted every day preferably for one week or longer, more preferably for two weeks or longer. The method of the invention may further contain a step of applying radiation therapy to a patient after the administration step.

The invention also provides arctigenin and/or arctiin for use in enhancement of an effect of radiation therapy. The invention also provides use of arctigenin and/or arctiin for the manufacture of a radiosensitizer for enhancing an effect of radiation therapy. The above descriptions for the anticancer agent, the radiosensitizer and the method of the invention can be applied to the embodiments of the arctigenin and/or arctiin of the invention and the embodiments of the use of the invention.

### EXAMPLES

Embodiments of the invention are explained in further detail below referring to Examples, but the invention is not limited to the following Examples.

### Example 1

A burdock fruit extract containing arctigenin was prepared by the following method.

After cutting burdock fruit (enzymatic activity of 7.82 U/g), the pieces which completely passed through a sieve of 9.5 mm were further passed through a sieve of 0.85 mm, and it was confirmed that 75% thereof remained. To 560 L of water which was kept at 30 to 32° C., 80 kg of the cut burdock fruit pieces were added, and the mixture was stirred for 30 minutes. Then, 253 L of ethanol was added, and the solution was heated to 85° C. and refluxed by heating for 40 minutes. The liquid was centrifuged, and the obtained liquid extract was obtained. This operation was repeated twice, and the obtained liquid extracts were combined and concentrated under reduced pressure. Dextrin was added at 25% to the solid contents of the extract, and the mixture was spray dried. The arctigenin and arctiin contents were 6.4% and 7.2%, respectively, and burdock fruit extract powder (containing 25% dextrin) with arctigenin/arctiin (weight ratio)=0.89 was thus obtained.

### Test Example 1

### (Test Method)

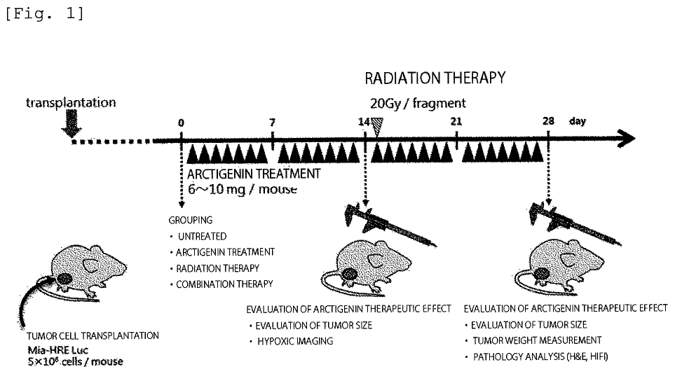

The antitumor effects of arctigenin treatment and radiation therapy were evaluated. The test method used in this Test Example is shown in FIG. 1.

To BALB/cAJc1-nu/nu mice (CLEA Japan, Inc.), 1×10⁶ human pancreatic cancer cells, MiaPaCa-2 cells were subcutaneously transplanted. On day 14 after the transplantation, the mice to which the cells were transplanted were divided into four groups of (a) untreated group, (b) arctigenin treatment group, (c) radiation therapy group and (d) arctigenin treatment-radiation therapy-combination group. The arctigenin treatment group and the combination group were treated with arctigenin for two weeks. The arctigenin treatment was conducted by giving feed containing 0.5% (w/w) of the burdock fruit extract of Example 1 to the mice. When the dose of arctigenin was calculated assuming that the daily intake of the feed of the mice was 3 to 5 g, the dose was 1.5 to 2.5 mg/individual/day. After two weeks of the arctigenin treatment, radiation was applied to the tumors of the radiation therapy group and the combination group using an x-ray irradiator (Faxitron X-ray (model: CP160)) under the setting conditions of 160 kV and 6.3 mA at a dose of 20 Gy/fraction through a copper plate filter. Then, the arctigenin administration group and the combination group were treated with arctigenin for two weeks. After the arctigenin treatment, the tumor sizes and the tumor weights of the groups were measured. In addition, the tumors were collected and pathologically analyzed.

### (Effects of Arctigenin Treatment)

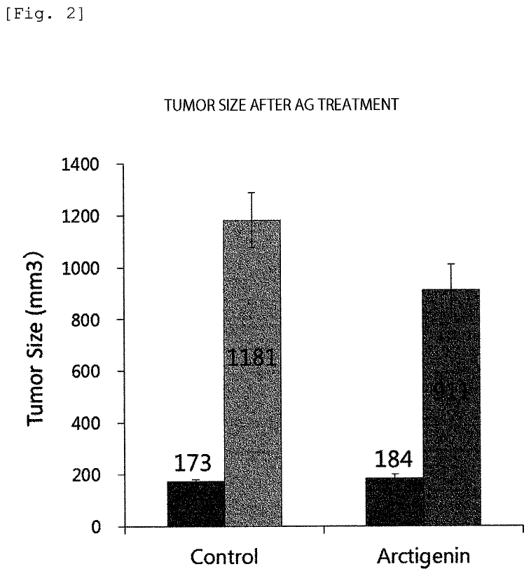

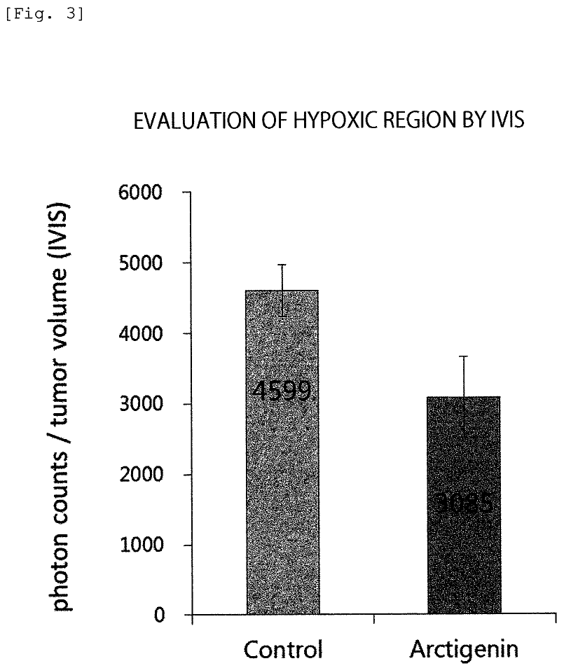

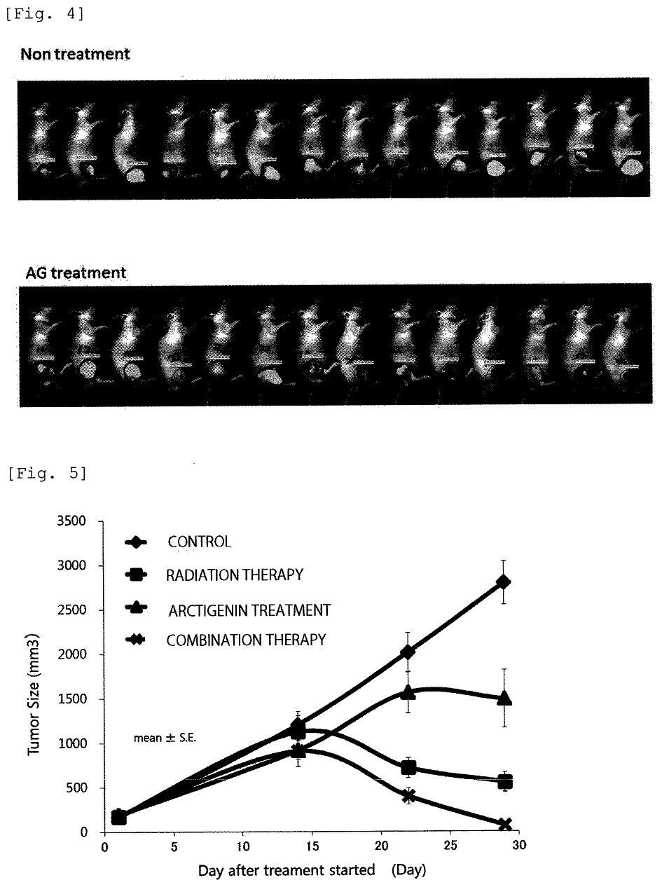

After two weeks of the first arctigenin treatment (before the radiation therapy), the average tumor sizes of the untreated group and the arctigenin treatment group were measured. The results are shown in FIG. 2. As shown in FIG. 2, the average tumor size of the arctigenin treatment group was significantly smaller than that of the untreated group. Thus, it was shown that the tumor size reduced significantly by the arctigenin treatment. In addition, the results of evaluation of the intratumoral hypoxic regions (photon counts/tumor volume) after the arctigenin treatment using IVIS are shown in FIG. 3. As shown in FIG. 3, the intratumoral hypoxic region of the arctigenin treatment group was significantly smaller than that of the untreated group. The results of IVIS imaging of the untreated group and the arctigenin treatment group are shown in FIG. 4. The results suggest that the hypoxic regions reduced significantly by the arctigenin treatment.

### (Change in Tumor Size)

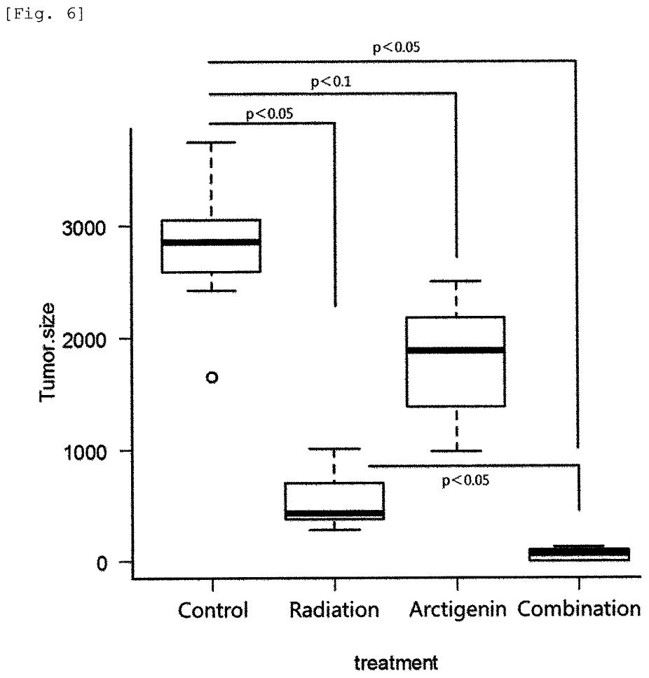

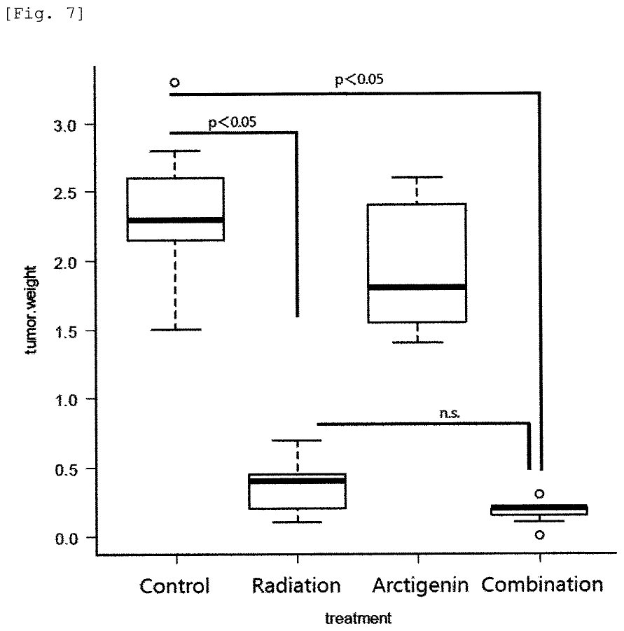

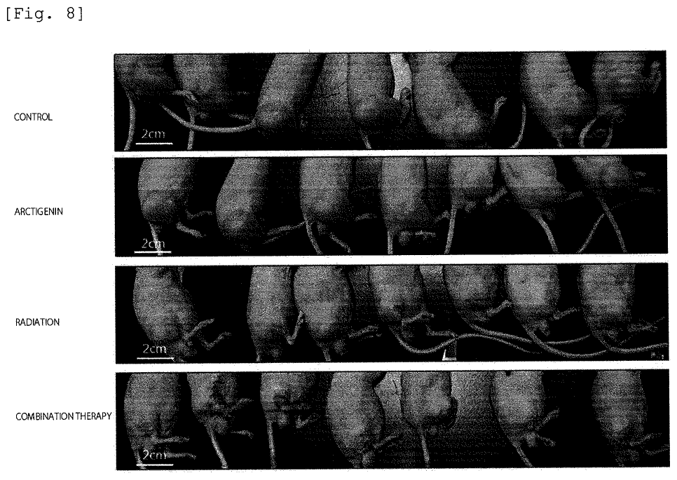

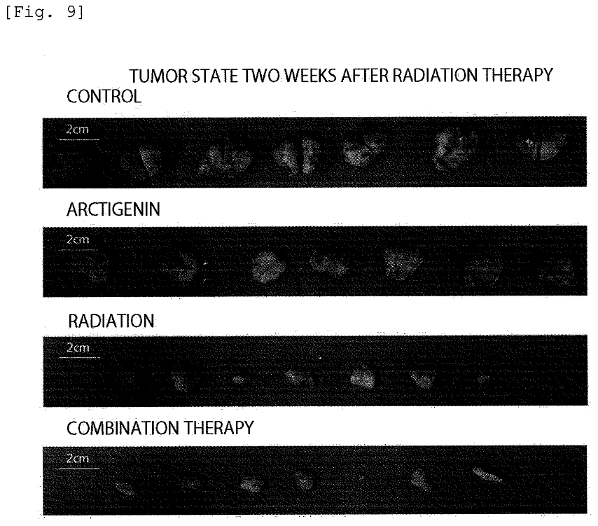

FIG. 5 is a graph showing the changes in average tumor size (mean±S.E.) of the treatment groups. The graph of FIG. 5 shows the average tumor sizes before starting the treatment (before grouping), two weeks after starting the treatment (after the first arctigenin treatment), one week after the radiation therapy and two weeks after the radiation therapy (after the second arctigenin treatment). FIG. 6 is a box plot showing the tumor sizes of the treatment groups after the end of the treatment (two weeks after the radiation therapy), and FIG. 7 is a box plot showing the tumor weights (g) of the treatment groups after the end of the treatment (two weeks after the radiation therapy). FIG. 8 and FIG. 9 are figures showing the tumor states of the treatment groups after the end of the treatment (two weeks after the radiation therapy).

As shown in FIG. 5 to FIG. 9, after the end of the treatment (two weeks after the radiation therapy), the tumor sizes and the tumor weights of the radiation therapy group (Radiation) and the combination group (Combination) were significantly smaller than those of the untreated group (Control), and there was a tendency towards a smaller tumor size in the arctigenin treatment group (Arctigenin). Moreover, it was found that the tumors became significantly small by the combination of the arctigenin treatment and the radiation therapy as compared to the case of the radiation therapy alone.

### Test Example 2

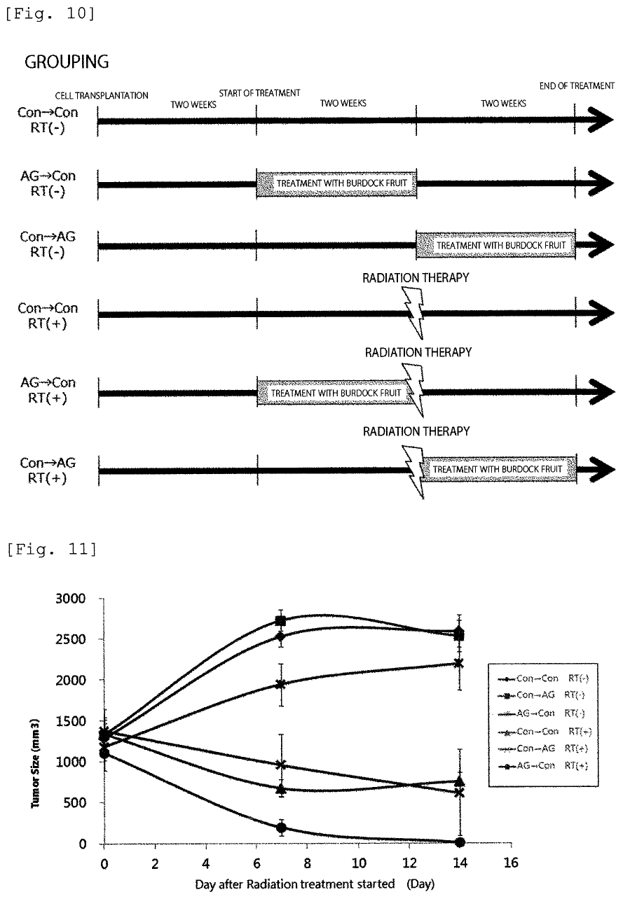

In order to evaluate whether the order of the burdock fruit extract treatment and the radiation therapy would affect the antitumor effects, arctigenin treatment was conducted for two weeks either before or after conducting radiation therapy. The test method used in this Test Example is shown in FIG. 10.

To BALB/cAJc1-nu/nu mice (CLEA Japan, Inc.), 1×10⁶ human pancreatic cancer cells, MiaPaCa-2 cells were subcutaneously transplanted. On day 14 after the transplantation, the mice to which the cells were transplanted were divided into (a) untreated group, (b) arctigenin treatment group, (c) radiation therapy group and (d) arctigenin treatment-radiation therapy-combination group. The (b) arctigenin treatment group was further divided into a group in which two-week arctigenin treatment was started 14 days after the transplantation and a group in which two-week arctigenin treatment was started four weeks after the transplantation. The (d) combination group was further divided into a group which received two-week arctigenin treatment before conducting the radiation therapy and a group which received two-week arctigenin treatment after conducting the radiation therapy.

The arctigenin treatment was conducted by giving feed containing 0.5% (w/w) of the burdock fruit extract of Example 1 to the mice. When the dose of arctigenin was calculated assuming that the daily intake of the feed of the mice was 3 to 5 g, the dose was 1.5 to 2.5 mg/individual/day. In the radiation therapy, radiation was applied to the tumors using an x-ray irradiator (Faxitron X-ray (model: CP160)) under the setting conditions of 160 kV and 6.3 mA at a dose of 20 Gy/fraction through a copperplate filter. After the two-week arctigenin treatment after the radiation therapy, the tumor sizes and the tumor weights of the groups were measured. In addition, the tumors were collected and pathologically analyzed.

(Changes in Tumor Size of Treatment Groups after End of Treatment)

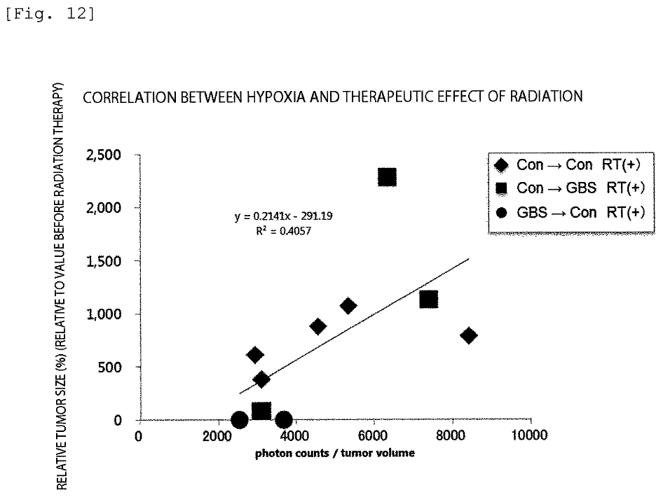

FIG. 11 is a graph showing the changes in average tumor size of the treatment groups. The graph of FIG. 11 shows the average tumor sizes of the treatment groups before starting the radiation therapy (two weeks after the cell transplantation), one week after the radiation therapy and two weeks after the radiation therapy. As shown in FIG. 11, the tumors shrank significantly in the combination group which received the arctigenin treatment before the radiation therapy (AG-Con, RT(+)) as compared to the combination group which received the arctigenin treatment after the radiation therapy (Con-AG, RT(+)).

FIG. 12 is a figure showing the correlation between the intratumoral hypoxic region after the end of the treatment (two weeks after the radiation therapy) evaluated using IVIS and the tumor size (%) two weeks after the radiation therapy relative to the tumor size before the radiation therapy. In FIG. 12, the results of the group which received the radiation therapy only (Con-Con, RT(+)), the group which received the arctigenin treatment after the radiation therapy (Con-GBS, RT(+)) and the group which received the arctigenin treatment before the radiation therapy (GBS-Con, RT(+)) are shown. As shown in FIG. 12, it is obvious that the effect of the radiation therapy of reducing the tumor size is stronger, as the hypoxic region in the tumor is smaller.

The results suggest that, by conducting the arctigenin treatment before the radiation therapy, the hypoxic state in the tumor was improved, and the environment changed into an environment in which the effects of the radiation therapy were exhibited more easily.

### INDUSTRIAL APPLICABILITY

The invention can heighten the effects of radiation therapy and thus can be suitably used as an anticancer agent, a radiosensitizer and a food composition which are used in combination with radiation therapy.

1. A radiosensitizer containing arctigenin and/or arctiin as an active ingredient.

2. A food composition for enhancing an effect of radiation therapy containing arctigenin and/or arctiin as an active ingredient.

3. The food composition according to claim 2, wherein the arctigenin and/or arctiin is contained as burdock, burdock fruit, burdock sprout, forsythia or an extract extracted from these.

4. A pretreatment agent for radiation therapy containing arctigenin and/or arctiin as an active ingredient.
